SEQUENCE LISTING (9 sequences)

SEQ ID NO: 1 - DNA, 27 bp, Artificial Sequence
  oligonucleotide
caaagaagcyttttcactatttgacaa

SEQ ID NO: 2 - DNA, 26 bp, Artificial Sequence
  oligonucleotide
tcatttttcttgccatcattgtcaga

SEQ ID NO: 3 - DNA, 20 bp, Artificial Sequence
  oligonucleotide
ggrgactactaccgycaggg

SEQ ID NO: 4 - DNA, 22 bp, Artificial Sequence
  oligonucleotide
caatctcccacatbgtcacccc

SEQ ID NO: 5 - DNA, 25 bp, Artificial Sequence
  oligonucleotide
ctattgtcgaccatacctctaccgt

SEQ ID NO: 6 - DNA, 20 bp, Artificial Sequence
  oligonucleotide
cgtggccgttgctggataag

SEQ ID NO: 7 - protein, 149 aa, H. sapien [sequence omitted]

SEQ ID NO: 8 - protein, 894 aa, H. sapien
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 9 - protein, 192 aa, Trichoplusia
QAQGSVTLNSDGSMGLGAKVPIVGNEKNVLSALGSVDLNDQLKPASRGMGLALDNVNGHGLSVMKETVPGFGDRLTGAGRVNVFHNDNHDISAKAFVTKNMPDFPNVPNFNTVGGGVDYMYKNKVGASLGMANTPFLDRKDYSAMGNLNVFRSPTTSVDFNAGFKKFDTPVFKSNWEPNFGLTFSRSFGNKW

(19) United States
(12) Patent Application Publication
(10) Pub. No.: US 2012/0141988 A1
(43) Pub. Date: Jun. 7, 2012

(54) METHOD OF CELL-LINE IDENTIFICATION

(76) Inventors: Jean-Pol Cassart, Rexensart (BE); Patricia Anne-Laure Lienard, Gosselies (BE)

(21) Appl. No.: 13/202,943

(22) PCT Filed: Feb. 19, 2010
(86) PCT No.: PCT/EP2010/052117

§ 371 Date: Nov. 11, 2011

(30) Foreign Application Priority Data
Feb. 23, 2009 (GB) 0903026.3

(51) Int. Cl. C12Q 1/68

(52) U.S. Cl. 435/6.11; 435/6.17; 435/6.12

(57) ABSTRACT

The present invention discloses a method of cell-line identification comprising one or more of the following steps: (a) analysis of the calmodulin gene; (b) analysis of the Axl receptor tyrosine kinase gene; (c) analysis of an attacin gene; or a combination thereof.

### FIELD OF THE INVENTION

The present invention relates to the field of the identification of cell-lines. More particularly it relates to identification of cell-lines through analysis by molecular techniques, and identifying variation in introns or determining whether a particular gene is present. This method is particularly useful for the quality control of cell-lines used for the production of pharmaceutical preparations.

### BACKGROUND OF THE INVENTION

Current methods performed in Quality Control (QC) use the isoenzyme technique. This method is based on the analysis of the electrophoretic mobilities of isoenzymes which differ between species. Mobility profiles can be established for each species using the combined analysis of the migration distances of enzymes such as: glucose-6-phosphate dehydrogenase (G6PD), aspartate aminotransferase (AST), lactate dehydrogenase (LDH) and malate dehydrogenase (MD). This method provides a means of determining the species of origin of a cell line and detecting cells cross-contaminated with cells from another cell line of different species (Stacey and Hockley, 2006). However, this test is labour intensive and time consuming.

### SUMMARY OF THE INVENTION

The present inventors have demonstrated that cell-lines can be identified by a method more reliably and easily than current identification methodologies. The identification method of the present invention is based on the analysis of the size of intron sequences which differ between species. This method involves the amplification of the intron sequences by polymerase chain reaction (PCR). The present inventors have identified genes that are sufficiently conserved in the exon sequences to allow the annealing with the amplification PCR primers and sufficiently different in the intron sequence to allow discrimination based on the length polymorphism. Discrimination of these variations can thus be determined through analysis of these genomic regions.

The method allows identification of a number of cell-line types derived from vertebrates (e.g. MRC-5, Vero, CHO, FRHL-2, MDCK, Chicken Embryo Fibroblasts [CEF]), based on informative polymorphic introns found in two genes, calmodulin and Axl receptor tyrosine kinase. Further, using a gene specific to insect cell-lines, analysis to determine the presence or absence of an insect gene can help identify insect derived cell-lines, for example Hi-5.

Accordingly, the present invention provides a method of cell-line identification comprising one or more of the following steps: (a) analysis of the calmodulin gene; (b) analysis of the Axl receptor tyrosine kinase gene; (c) analysis of an attacin gene; or a combination thereof.

### BRIEF DESCRIPTION OF FIGURES

FIG. 1 Analysis of the CALM3/4 PCR products.

FIG. 2 Analysis of the AXL 18/19 PCR products.

FIG. 3 Analysis of the PPATTA PCR product.

### DESCRIPTION OF THE INVENTION

The terms “comprising”, “comprise” and “comprises” herein are intended by the inventors to be optionally substitutable with the terms “consisting of”, “consist of”, and “consists of”, respectively, in every instance.

The present inventors have identified both conserved and variable regions in the genes encoding Calmodulin and Axl receptor tyrosine kinase. The variation in the genes allows for discrimination between the different cell-lines; these variations can be detected using any method that allows analysis of the nucleic acid. Furthermore, the conserved regions can be used to design complementary primers for either PCR amplification or sequencing reaction that can be used to analyse cell-lines from many sources. Insect-derived cell lines can be identified by detecting insect specific nucleotide sequences.

Accordingly, the present invention provides a method of cell-line identification comprising one or more of the following steps: (a) analysis of the calmodulin gene; (b) analysis of the Axl receptor tyrosine kinase gene; (c) analysis of an attacin gene; or a combination thereof.

The present invention may be used to identify many types of cell-lines. The term “cell-line” as used herein refers to established cell-cultures grown in vitro. Cell-lines may be derived from any species of animal including mammals, birds and insects. In a particular embodiment the identification method of the present invention is used for cell lines used in the pharmaceutical industry. In a particular embodiment, the methods of the present invention are used to identify cell-lines used in the production of viruses that may be used in vaccines. The term “cell-line” encompasses continuous cell-cultures which are cell-cultures of morphologically uniform cells that can be propagated in vitro for an indefinite time. Accordingly, in one embodiment, methods of the invention can be used to identify and/or differentiate cell-lines selected from the group: MRC-5, Vero, Hi-5, CHO, FRHL2, and MDCK. The term “cell-line” also incorporates primary cell-lines, i.e. cells that are isolated directly from a subject. Accordingly, in one embodiment, the methods of the present invention are used to identify chicken derived cells, for example, CEF (chicken embryo fibroblasts).

The present inventors have identified genes calmodulin and Axl receptor tyrosine kinase which comprise both conserved exons and variable introns. Accordingly, in one embodiment of the invention, there is provided a method of cell-line identification comprising the step: analysis of the calmodulin gene. In a further embodiment of the invention there is provided a method of cell-line identification comprising the step: analysis of the Axl receptor tyrosine kinase gene.

An insect specific gene has also been identified, allowing discrimination between insect derived cells and cells derived from other organisms, for example mammalian or avian cells. Accordingly, in a further embodiment of the invention there is provided a method of cell-line identification comprising the step: analysis of an attacin gene.

The term “analysis” refers to any analytical technique that will allow the skilled person to differentiate/discriminate nucleic sequences from different cell lines. There are a large number of techniques suitable for this purpose known to the skilled person, these techniques include but are not limited to: Polymerase Chain Reaction (PCR) amplification, sequencing, hybridisation, and restriction length fragment polymorphisms (RFLP). In one embodiment of the invention, there is provided a cell-line identification method of the invention wherein the analysis method is selected from the group consisting of Polymerase Chain Reaction (PCR) amplification, sequencing, hybridisation and restriction length fragment polymorphisms (RFLP).

The term “calmodulin gene” is well known to the person skilled in the art and as used herein is intended to mean a gene that encodes a polypeptide comprising at least one EF-hand motif that is capable of binding calcium. Calmodulin plays a major role in transmitting intracellular Ca²⁺ signals to a wide variety of effector systems (Rhyner et al., (1994) Euro J Biochem 225: 71-81). In mammals, 3 genes (CALM1, CALM2 and CALM3) encode the single, highly conserved, calmodulin (CaM) protein (Berchtold et al., (1993) Genomics 16: 461-465). In particular embodiments of the invention the term calmodulin gene is a nucleic acid sequence that encodes a polypeptide of SEQ ID NO: 7 or encodes an amino acid sequence having 70%, 75%, 80%, 85%, 90%, 95%, 96%, 97%, 98% or 99% sequence identity to SEQ ID NO:7.

The term “attacin gene” as used in herein refers to nucleic acid sequences that encode insect antibacterial peptides known as attacins and attacin-like proteins, including basic and acidic attacins (see for example Boman et al., (1991) Eur J. Biochem 201: 21-31). The term encompasses genomic sequences that encode attacins, proattacins and pre-proattacins; these terms are well known to those skilled in the art. Attacins are polypeptides that are specific to insects and are thus not found in mammals and or birds. In particular embodiments of the invention the term attacin gene is a nucleic acid sequence that encodes a polypeptide of SEQ ID NO: 9 or polypeptides with amino acid sequences that have 70%, 75%, 80%, 85%, 90%, 95%, 96%, 97%, 98% or 99% sequence identity to SEQ ID NO: 9.

The term “Axl receptor tyrosine kinase” is well know to the person skilled in the art and is used herein to mean transmembrane proteins involved in intracellular signalling which is triggered through activation of the tyrosine kinase domain and the subsequent phosphorylation of a variety of substrates and which is a tyrosine kinase receptor part of the Axl family. Axl receptor tyrosine kinases are ubiquitously expressed in a number of cell lines including those of epithelial, mesenchymal and haematopoietic origin, as well as non-transformed cells (Hafizi & Dahlhbäck, 2006 [Cytokine & Growth Factor Reviews 17: 295-304]).

The transforming activity of the Axl receptor tyrosine kinase demonstrates that the receptor can drive cellular proliferation. Although the function of the Axl receptor tyrosine kinase in nontransformed cells and tissues was unknown, Varnum et al. (1995, Nature, 373: 623-626) suspected that it may involve the stimulation of cell proliferation in response to an appropriate signal, i.e., a ligand that activates the receptor. Varnum et al. (1995) purified an Axl receptor tyrosine kinase stimulatory factor and identified it as the product of the growth arrest-specific gene-6 (GAS6). The Axl tyrosine kinase gene encodes a tyrosine kinase receptor which regulates cell growth and differentiation (O'Bryan et al., (1991) Mol Cell Biol 11: 5016-31). The genomic organization of the human Axl tyrosine kinase gene locus was described by Schulz et al., (1993 Oncogene 8: 509-13). Human Axl tyrosine kinase gene contains 20 exons that are distributed over a region of 44 kb.

In particular embodiments of the invention the term Axl receptor tyrosine kinase gene means a nucleic acid sequence that encodes a polypeptide of SEQ ID NO: 8 or polypeptides with amino acid sequences that have 70%, 75%, 80%, 85%, 90%, 95%, 96%, 97%, 98% or 99% sequence identity to SEQ ID NO: 8.

In a further embodiment, there is provided a method of cell-line identification comprising the following steps: (a) analysis of the calmodulin gene; and (b) analysis of the Axl receptor tyrosine kinase gene.

In a further embodiment, there is provided a method of cell-line identification comprising the following steps: (a) analysis of the calmodulin gene; and (b) analysis of an attacin gene.

In one embodiment, there is provided a method of cell-line identification comprising the following steps: (a) analysis of an attacin gene; and (b) analysis of the Axl receptor tyrosine kinase gene.

In a further embodiment of the invention there is provided a method of cell-line identification comprising the following steps: (a) analysis of the calmodulin gene; (b) analysis of the Axl receptor tyrosine kinase gene; and (c) analysis of an attacin gene.

It is envisaged that the skilled person may wish to use a variety of molecular techniques in order to identify one or more cell lines. For example, in certain embodiments of the invention the skilled person may use one or more or any combination of the following techniques: PCR, sequencing, hybridisation and RFLP. Accordingly, the methods of the invention may be performed using one or more of the molecular techniques selected from the list comprising PCR, sequencing, hybridisation and RFLP, or any combination thereof.

In a particular embodiment of the invention there is provided a method of cell-line identification comprising the steps comprising one or more of the following steps: (a) PCR amplification of a calmodulin gene or fragment thereof; (b) PCR amplification of a Axl receptor tyrosine kinase gene or fragment thereof; or (c) PCR amplification of an attacin gene or fragment thereof; or any combination thereof.

The term “fragment” as herein described refers to any nucleic acid sequence of a gene, that is not the whole gene sequence, comprising at least 50, 60, 70, 80, 90, 100, 110, 120, 130, 140, 150, 160, 170, 180, 190, 200, 400, 600, 800, 1000, 1200, 1400, 1600 or more nucleotides.

In a further aspect of the invention there is provided a method of cell-line identification comprising one or more of the following steps: (a) hybridisation of a probe to the calmodulin gene or fragment thereof; (b) hybridisation of a probe to an Axl receptor tyrosine kinase gene or fragment thereof; or (c) hybridisation of a probe to an attacin gene or fragment thereof; or any combination thereof.

In one aspect of the invention there is provided a method of cell-line identification comprising one or more of the following steps: (a) sequencing of the calmodulin gene or fragment thereof; (b) sequencing of an Axl receptor tyrosine kinase gene or fragment thereof; or (c) sequencing of an attacin gene or fragment thereof; or any combination thereof.

The genes encoding calmodulin comprise introns that vary in size between different cell-lines. Accordingly, in an embodiment, the cell-line is indentified by the size (number of nucleotides) of the amplicon (the amplified product following PCR). The inventors have determined that introns within the calmodulin and Axl receptor tyrosine kinase genes vary in size between different species and/or strains. Accordingly, in a particular embodiment of the invention cell-lines can be differentiated and/or determined by the size of a particular intron between any two exons. In a particular embodiment of the invention, cell-lines are determined by the size of the amplicon derived from either the calmodulin gene and/or the Axl receptor tyrosine kinase gene.

In one embodiment there is provided a method of cell-line identification comprising the step: PCR amplification of the calmodulin gene or fragment thereof.

PCR amplification of a fragment of the calmodulin gene allows identification and/or differentiation of cell-lines as the same genes from different cell-lines differ in the length of introns. Thus primer sets that are designed to amplify one or more of the introns found in the calmodulin gene can be used to identify and/or differentiate cell-lines. Accordingly, in one embodiment of the invention there is provided a method of cell-line identification comprising the step: PCR amplification of one or more of the calmodulin introns or a fragment thereof. In a further embodiment, there is provided a method of cell-line identification comprising the step: PCR amplification of the intron between exons 3 and 4 of the calmodulin gene.

In one embodiment of the invention there is provided a method wherein a fragment of the calmodulin gene is amplified by PCR and in a further embodiment of the invention there is provided a method wherein the amplified fragment comprises the sequence between exon 3 and exon 4 of the calmodulin gene.

In a further embodiment of the invention there is provided a method wherein a fragment of the calmodulin gene is amplified using at least one of the primers selected from SEQ ID NO: 1 and SEQ ID NO: 2 or functional derivative thereof.

The Axl receptor tyrosine kinase gene has conserved exons and introns that vary between different cell-lines. Accordingly, in one embodiment there is provided a method of cell-line identification comprising the step: PCR amplification of an Axl receptor tyrosine kinase gene or fragment thereof.

PCR amplification of a fragment of the Axl receptor tyrosine kinase gene allows differentiation between cell-lines as different cell-lines differ in the length of introns. Thus primer sets that are designed to amplify one or more of the introns found in Axl receptor tyrosine kinase genes can be used to differentiate between differing cell-lines; thus amplification of an intron that varies in length between different cell-lines falls within the scope of the invention. Accordingly, in one embodiment of the invention there is provided a method of cell-line identification comprising the step: PCR amplification of one or more Axl receptor tyrosine kinase gene introns or fragment thereof. In a further embodiment, there is provided a method of cell-line identification comprising the step: PCR amplification of the intron between exons 18 and 19 of Axl receptor tyrosine kinase gene.

In one embodiment of the invention there is provided a method a fragment of the Axl receptor tyrosine kinase gene is amplified by PCR. In a further embodiment of the invention there is provided a method wherein the amplified fragment comprises the sequence between exon 18 and exon 19 of the Axl receptor tyrosine kinase gene. Suitably, the method wherein a fragment of the Axl receptor tyrosine kinase gene is amplified uses at least one of the primers selected from SEQ ID NO: 3 and SEQ ID NO: 4 or functional derivative thereof.

The cell-type can be determined by the presence or absence of the gene i.e. the particular cell-line may be differentiated from another through either the presence or absence of a particular gene or fragment thereof.

By “presence” of a gene it is meant that the whole or part of the specified gene is detectable by methods well known to those skilled in the art.

For example, an insect cell-line can be differentiated from a mammalian cell-line through the detection of a gene only present in insect cells.

Accordingly, in one embodiment of the invention there is a provided a method of cell-line identification comprising the step wherein the cell-line type is determined by the presence and/or absence of an attacin gene or fragment thereof.

Accordingly, in one embodiment of the invention there is provided a method of cell-line identification comprising the step: PCR amplification of the pre-proattacin (PPATT) gene or fragment thereof. In a further embodiment of the invention, there is provided a method of cell-line identification comprising the step: PCR amplification of the pre-proattacin A (PPATTA) gene or fragment thereof.

In one embodiment of the invention there is provided a method wherein a fragment of the attacin gene is amplified by PCR. Accordingly, a method wherein a fragment of the PPATT gene is amplified using at least one of the primers comprising the sequences selected from SEQ ID NO: 5 and SEQ ID NO: 6 or functional derivative thereof.

However, it is clear to the person skilled in the art that the presence and or absence of a gene can be determined by a number of techniques including PCR amplification of the gene or part thereof. Other techniques by which the presence of genes may be assessed include but are not limited to DNA hybridisation, including in situ DNA hybridisation. As used herein, the terms “hybridisation” or “specific hybridisation” means that the primer or probe forms a duplex (double-stranded nucleotide sequence) with part of a target or with the entire region under the experimental conditions used, and that under those conditions the primer or probe does not form a duplex with other regions of the nucleotide sequence present in the sample to be analysed. It should be understood that the primers and probes of the present invention are designed for specific hybridisation to specific genes and may therefore fall entirely within said region or may to a large extent overlap with said region (i.e. form a duplex with nucleotides outside as well as within said region). Accordingly, there is provided a method in which the primers as herein described may be used during a PCR reaction or DNA hybridisation.

Polymerase Chain Reaction (PCR) amplification maybe performed from the genomic nucleic acid isolated and or purified from a cell-culture. Encompassed within the invention are PCR reactions carried out on a cell suspension, crude, isolated or purified genomic preparation, using techniques of genomic DNA preparation well known to those skilled in the art. The genomic preparations encompassed by the present invention may be isolated or substantially purified. By “isolated” or “substantially purified” is intended that the nucleic acid molecules, are substantially or essentially free from components normally found in association with the nucleic acid in its natural state. Such components include other cellular material and culture media for example.

Methods for designing PCR primers are generally known in the art and are disclosed in Sambrook and Russel, Molecular Cloning: A Laboratory Manual (Cold Harbour Laboratory Press). Known methods of PCR include, but are not limited to, methods using paired primers, nested primers, single specific primers, degenerate primers, gene-specific primers, vector-specific primers, partially mismatched primers, and the like.

With PCR, it is possible to amplify a single copy of a specific target sequence to a level detectable by several different methodologies (e.g., but not limited to, hybridization with a labelled probe; incorporation of biotinylated primers followed by avidin-enzyme conjugate detection; incorporation of 32P-labeled deoxynucleotide triphosphates, such as dCTP or dATP, into the amplified segment, incorporation of a fluorochrome such as etidium bromide, or other commercial compounds). In addition to genomic DNA, any nucleotide sequence can be amplified with the appropriate set of primer molecules. In particular, the amplified segments created by the PCR process itself are, themselves, efficient templates for subsequent PCR amplifications.

Amplification in PCR requires “PCR reagents” or “PCR materials,” which herein are defined as all reagents necessary to carry out amplification except the polymerase, primers, and template. PCR reagents normally include nucleic acid precursors (dCTP, dTTP, etc.), and buffer.

The term “primer” is used herein to mean any single-stranded oligonucleotide sequence capable of being used as a primer in, for example, PCR technology. Thus, a ‘primer’ according to the invention refers to a single-stranded oligonucleotide sequence that is capable of acting as a point of initiation for synthesis of a primer extension product that is complementary to the nucleic acid strand to be copied. The design (length and specific sequence) of the primer will depend on the nature of the DNA and/or RNA targets and on the conditions at which the primer is used (such as temperature and ionic strength).

The primers may consist of the nucleotide sequences shown in SEQ ID NO: 1 to 6, or may be 1, 2, 3, 4, 5, 6, 7, 8, 9 10, 15, 20, 25, 30, 35, 40, 45, 50, 75 or 100 or more bases which comprise or fall within the sequences of SEQ ID NO: 1 to 6, provided they are suitable for specifically binding DNA of target loci, under stringent conditions. When needed, slight modifications of the primers or probes in length or in sequence can be carried out to maintain the specificity and sensitivity required under the given circumstances and thus under 1, 2, 3, 4, 5, 6 or more nucleotides may be substituted. Probes and/or primers listed herein may be extended by 1, 2, 3, 4 or 5 nucleotides, for example, in either direction.

For the avoidance of doubt one letter designation other than A, T, G and C mean the following:

Single letter code

B = C or G or T
D = A or G or T
H = A or C or T
K = G or T
M = A or C
N = A or C or G or T
R = A or G
S = C or G
V = A or C or G
W = A or T
Y = C or T

The term “functional derivative” is used herein to mean a primer that comprises a sequence that is at least 95% identical to a primer as defined herein over the length of the primer, suitably greater than 95% identical such as 96%, 97%, 98%, 99% and most preferably has 100% identity over its length. Functional derivatives may have 1, 2, 3, 4, 5, 6, 7, 8, 9 or 10 base deletions at the 5′ end or 3′ end or both.

As used herein, the term “stringent conditions” means any hybridisation conditions which allow the primers to bind to a specific nucleotide sequence, and not to any other loci on the cell genome. Such stringent conditions are known to those skilled in the art and can be found in Current Protocols in Molecular Biology, John Wiley & Sons, N.Y. (1989), 6.3.1-6.3.6. A non-limiting example of stringent hybridization conditions is hybridization at 6× sodium chloride/sodium citrate (SSC) at about 45° C., followed by one or more washes in 0.1×SSC, 0.2% SDS at about 68° C. An alternate example of stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.).

The methods of the invention can be adapted according to the cell-lines that are required to differentiated or determined. For example, some cell-lines are capable of being differentiated by PCR amplification of an intron of calmodulin alone. However, some cell-lines may result in an amplicon that is the same size or as another cell-line and thus further steps are required for cell-line identification.

Accordingly, in one embodiment there is provided a cell-line identification method comprising the steps: PCR amplification of the calmodulin gene or fragment thereof.

In a further embodiment of the invention there is provided a cell-line identification method comprising the steps: PCR amplification of the attacin gene or fragment thereof.

In a further embodiment of the invention there is provided a cell-line identification method comprising the steps: PCR amplification of the Axl receptor tyrosine kinase gene or fragment thereof.

In a further embodiment there is provided a cell-line identification method comprising the following steps: (a) PCR amplification of the calmodulin gene or fragment thereof; and (b) PCR amplification of an attacin gene or fragment thereof.

In a further embodiment there is provided a cell-line identification method comprising the following steps: (a) PCR amplification of the calmodulin gene or fragment thereof; and (b) PCR amplification of the Axl receptor tyrosine kinase gene or fragment thereof.

In a further embodiment there is provided a cell-line identification method comprising the following steps: (a) PCR amplification of an attacin gene or fragment thereof; and (b) PCR amplification of an Axl receptor tyrosine kinase gene or fragment thereof.

In one embodiment there is provided a cell-line identification method comprising the following steps: (a) PCR amplification of the calmodulin gene or fragment thereof; (b) PCR amplification of an attacin gene or fragment thereof; and (c) PCR amplification of the Axl receptor tyrosine kinase gene or fragment thereof.

In a particular embodiment of the invention, the PCR reactions may be performed in a single PCR reaction. Accordingly, one of embodiment of the invention provides a method in which the PCR amplification reactions are multiplexed. As used herein, the term “multiplexed” means any number of PCR reaction are performed in a single reaction tube i.e. the contents of a single reaction tube comprises more than 1 set of primers. Primers of the invention are designed so that a single PCR reaction can be performed using specified reaction conditions allowing amplifications of each gene or fragment thereof used in the cell-identification method (Calmodulin, Axl receptor tyrosine kinase and attacin) without prejudice to the amplification of another gene. Multiplexing allows a faster and easier method of molecular identification of cell-line as described herein.

The methods of the invention are suitable for the identification of a variety of cell-lines comprising a calmodulin, Axl receptor tyrosine kinase or attacin gene. In one embodiment of the invention there is provided a method for identification cell-lines used in the pharmaceutical industry, particularly I the production of vaccines. In one embodiment of the invention there is provided a method for identification cell-lines MRC-5, Vero, Hi-5, CHO, FRHL2, CEF and MDCK. These cell-lines are well known to those skilled in the art, however to summarise:

| Cell line | Organism |
|---|---|
| CHO | Cricetulus griseus |
| | Chinese Hamster |
| MRC-5 | Homo sapiens |
| | Human |
| MDCK | Canis familiaris |
| | Dog |
| Vero | Cercopithecus aethiops |
| | African Green Monkey |
| FRHL2 | Macaca mulatta |
| | Rhesus Monkey |
| Chicken | Gallus gallus |
| Hi-5 | Trichoplusia ni |
| | Lepidoptera Insect |

In a further embodiment, there is provided a kit for cell-line identification comprising primers complementary to any one of: the calmodulin gene; an Axl receptor tyrosine kinase gene; or an attacin gene; or any combination thereof. In a further embodiment the kit comprises at least one primer comprising or consisting of the following sequences or functional derivative thereof: SEQ ID NO: 1, SEQ ID NO: 2, SEQ ID NO: 3, SEQ ID NO: 4, SEQ ID NO: 5 or SEQ ID NO: 6 or functional derivative thereof.

In one embodiment of the invention there is provided the use of a calmodulin gene, a tyrosine kinase gene and/or the attacin gene or any combination thereof in cell-line identification.

### EXAMPLES

### 1.1 Extraction of Genomic DNA and PCR Amplification

Genomic DNA was extracted from CHO, MRC-5, MDCK, VERO, FRHL2, Hi-5 and Gallus [CFO] cells using the High Pure PCR template Preparation Kit (Roche). PCR was then performed on the under the following reaction conditions using the primers Calm ex 3F (SEQ ID NO:1), Calm ex 4R (SEQ ID NO:2), Axl ex 18F (SEQ ID NO:3), Axl 19R (SEQ ID NO:4), ppatta F (SEQ ID NO:5) and ppatta R (SEQ ID NO:6):

| Mix | | Cycles | | |
|---|---|---|---|---|
| Volume (μl) | Ingredients | Temperature | Time | Cycles |
| to 25 | H₂O_{PCR} | 95° C. | 4 min | |
| 2.5 | Tp PCR 10x (+MgCl₂) | 95° C. | 30 sec | X35 |
| 1 | dNTP 10 mM | 60° C. | 30 sec | |
| 0.2 | Primer Axl ex18F | 72° C. | 1 min | |
| 0.2 | Primer Axl ex19R | 72° C. | 7 min | |
| 0.1 | Primer Calm ex 3F | | | |
| 0.1 | Primer Calm ex 4R | | | |
| 0.05 | Primer ppatta F | | | |
| 0.05 | Primer ppatta R | | | |
| 0.3 | Ex Taq Takara | | | |
| 1 | DNA (100 ng) | | | |

### 2.1 Sizing of the PCR Products by Capillary Electrophoresis

The following example demonstrates that the difference in the size of the intron between exons 3 and 4 of calmodulin and 18 and 19 of Axl, can unequivocally identify the 7 different cell lines form which genomic DNA was extracted (see 1.1).

In order to allow the detection of the DNA products after capillary electrophoresis, the reverse primers used during the PCR amplification were labelled with distinct fluorescent dyes; the reverse primer for PPTTA was labelled with NED, the reverse primer for calmodulin was labelled with Hex and the Axl reverse primer was labelled with 6-FAM. The resulting labelled DNA fragments were run in parallel with a size marker, allowing the analysis of the PCR product with a size resolution around 1-bp for fragments of less than 500-bp.

Genomic DNA was amplified using fluorescent reverse primers at a hybridization temperature of 60° C. (according to the PCR conditions described above). The capillary electrophoresis was performed with the Genetic Analyzer instrument (ABI3730) using 50 cm capillaries (ROX-1200 ladder, ABI).

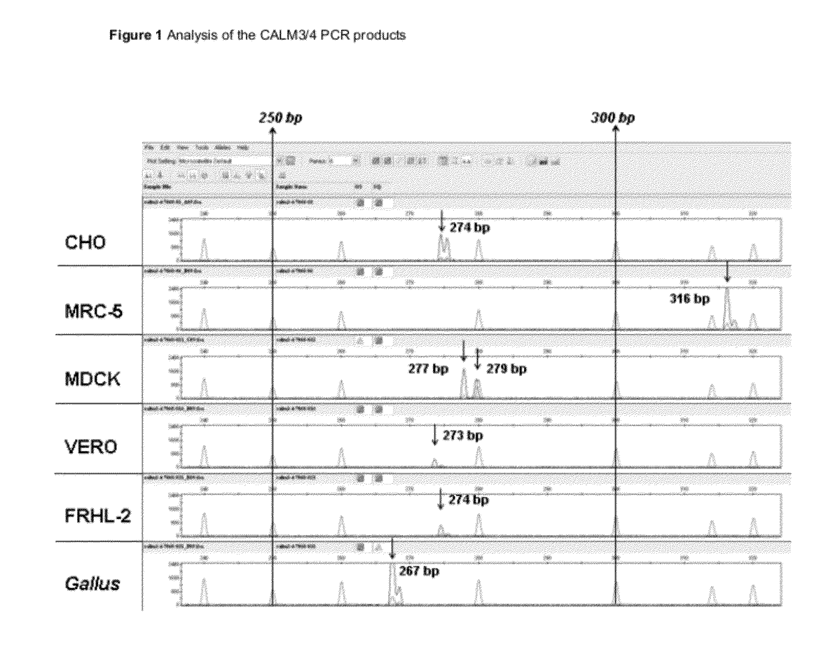

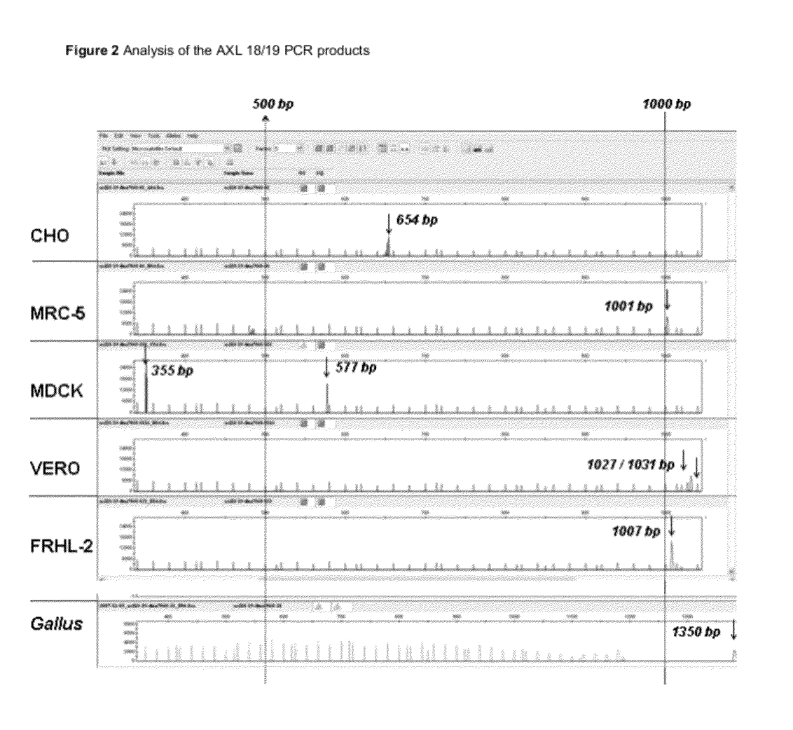

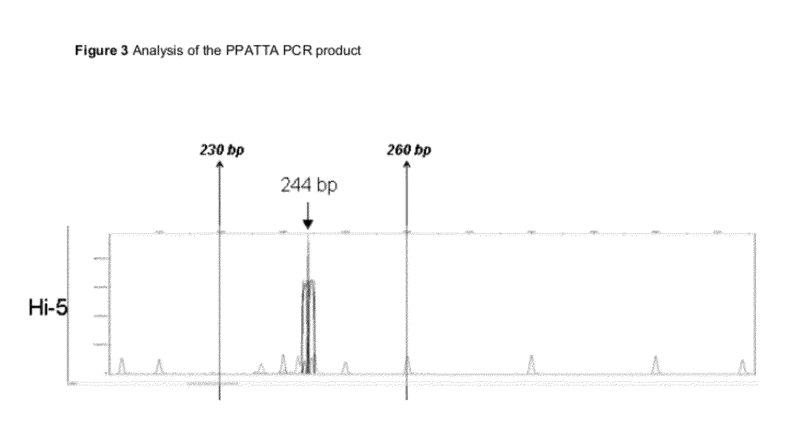

The sizes of the amplified products were compared to the expected sizes in Table 1. The capillary electrophoresis profiles are presented in FIGS. 1 to 3.

TABLE 1
Comparison of the PCR product sizes determined by capillary electrophoresis
(observed from the cells) with the sizes deduced from the published sequences
(expected from the organism).

| | Cells | | | | | |
|---|---|---|---|---|---|---|
| | PPATTA | | CALM3/4 | | AXL18/19 | |
| | | Expected | Observed | Expected | Observed | Expected |
| Size (bp) | Observed Cells | Organism | Cells | Organism | Cells | Organism |
| MRC-5 | NA | NA | 316 | 320 | 1001 | 1010 |
| VERO | NA | NA | 273 | ND | 1027 + 1031 | ND |
| CHO | NA | NA | 274 | ND | 654 | ND |
| FRHL2 | NA | NA | 274 | 278 | 1007 | 1021 |
| MDCK | NA | NA | 277 + 279 | 283 | 355 + 577 | 583 |
| Chicken | NA | NA | 267 | 270 | 1350 | 1489 |
| Hi-5 | 244 | 235 | NA | NA | NA | NA |

NA: Not applicable
ND: Not described

### CALM3/4 (Amplification of the Calmodulin Gene Between Exons 3 and 4) PCR Products

The size of the PCR products amplified from MRC-5, FRHL-2, MDCK and Chicken cells were very close to the predicted sizes. Two peaks were observed from MDCK cells (277 and 279-bp). A 274 bp fragment was amplified from CHO and FHRL-2 cells, close to the 273-bp fragment amplified from VERO cells. Other fragments were different by at least 3 base pairs. The sizes of the VERO and CHO fragments were not known before performing the experiment.

### AXL18/19 (Amplification of the Calmodulin Gene Between Exons 18 and 19) PCR Products

AXL 18/19 PCR allowed the discrimination of all 6 vertebrate DNAs. The size of the products amplified from VERO (1027 and 1031-bp) and CHO (654-bp) cells were clearly different.

### PPATTA (Attacin) PCR Products

The size of the DNA fragment amplified from T. ni DNA using the PPATTA primers was estimated at 244-bp. This size was matching with the expected 235-bp.

### Conclusions

The combined analysis of CALM3/4 and AXL18/19 PCR products yielded size patterns allowing the unequivocal identification of the 6 vertebrate cell lines.

### 3.1 Ability to Detect a Minor Species i.e. Contamination

The objective of the following example was to evaluate the capacity of the PCR assay above to detect a mix of 2 cell different types wherein the major species represents 90%, and the minor species represents 10% of the mix.

The 90/10% mixes of different cell types were performed at the level of the genomic DNA considering the number of genome copies (see Table 2)

The number of genome copies was calculated from the amount of DNA (measured by the Threshold system) and from the genome sizes (http://www.genomesize.com)

TABLE 2
Determination of the number of genome copies

| | | | Volume | Volume |
|---|---|---|---|---|
| | Haploid | | in 100 μl | in 100 μl |
| | genome | DNA | to obtain | to obtain |
| | size | concentration | 9000 copies/ | 1000 copies/ |
| Cell line | (pg) | (ng/μl) | μl | μl |
| MRC-5 | 3.5 | 83 | 37.8 | 4.2 |
| Vero | 3.5 | 58 | 54 | 6 |
| Hi-5 | 0.5 | 96 | 5 | 0.5 |
| CHO | 3.5 | 69 | 45 | 5 |
| FRHL2 | 3.5 | 126 | 25.2 | 2.8 |
| MDCK | 3.2 | 34 | 85.5 | 9.5 |
| Chicken | 1.25 | 38 | 29.7 | 3.3 |

Approximately 27,000 copies of the major species and 3,000 copies of the minor species were mixed and amplified under the PCR conditions described above (see 1.1)

### Results

The detection results from the different mixes are summarized in the Table 3.

TABLE 3
Detection of a minor species present at 10% in cell line mix-up

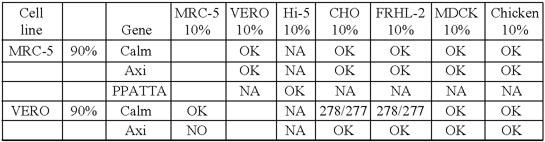

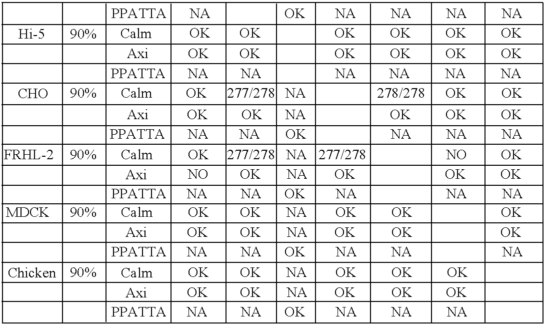

OK: 10% of the minor species was detected using the mentioned PCR
When the species cannot be discriminated because the size of the fragment are identical or very close, the size of both products are indicated in the table
NO: 10% of the minor species was not detected using the mentioned PCR
NA: Not applicable

For some mixes, a single PCR amplification did not allow to detect the minor species:
CHO/VERO/FRHL2 mixes could not be detected using the CALM3/4 PCR because the products displayed identical or similar sizes
10% MDCK in FRHL-2 could not be detected with the CALM3/4 assay
10% MRC5 in FRHL-2 and in VERO could not be detected with the AXL18/19 assay

It can thus be seen that the combined analysis of CALM3/4, AXL18/19 and PPATTA PCR products allowed the detection of a 10% DNA contamination between all different 7 cell lines, and that in some instances only 1 or 2 genes analyses were required to detect the contamination.

1. A method of identifying a test cell as having a reference cell-line comprising the following steps: (a) analyzing at least one gene from the test cell to determine the number of nucleotides of an intron of said gene, (b) comparing the number of nucleotides of the intron of the gene to the number of nucleotides of the intron of the gene known to be present in the reference cell-line, (c) identifying the test cell as being from the reference cell-line when the number of nucleotides in the introns being compared correspond; the gene being selected from the group of genes comprising a calmodulin gene, an Axl receptor tyrosine kinase gene and an attacin gene.

2. The method of claim 1 wherein the calmodulin gene is a nucleic acid sequence that encodes a polypeptide of SEQ ID NO: 7 or encodes an amino acid sequence having 70%, 75%, 80%, 85%, 90%, 95%, 96%, 97%, 98% or 99% sequence identity to SEQ ID NO:7.

3. The method of claim 1 wherein the Axl receptor tyrosine kinase gene is nucleic acid sequence that encodes a polypeptide of SEQ ID NO: 8 or polypeptides with amino acid sequences that have 70%, 75%, 80%, 85%, 90%, 95%, 96%, 97%, 98% or 99% sequence identity to SEQ ID NO: 8.

4. The method of any one of claim 1 wherein the attacin gene is a nucleic acid sequence that encodes a polypeptide of SEQ ID NO: 9 or polypeptides with amino acid sequences that have 70%, 75%, 80%, 85%, 90%, 95%, 96%, 97%, 98% or 99% sequence identity to SEQ ID NO: 9.

5. The method of claim 1 wherein the analyzing method is selected from the group consisting of Polymerase Chain Reaction (PCR) amplification, sequencing, hybridisation or restriction length fragment polymorphisms (RFLP), or any combination thereof.

6. (canceled)

7. The method claim 5 wherein the calmodulin gene or fragment thereof is amplified by PCR.

8. The method of claim 7 wherein the amplified fragment comprises the sequence between exon 3 and exon 4 of the calmodulin gene or part thereof.

9. The method of claim 5 wherein the Axl receptor tyrosine kinase gene or fragment thereof is amplified by PCR.

10. The method of claim 9 wherein the amplified fragment comprises the sequence between exon 18 and exon 19 of the Axl receptor tyrosine kinase gene.

11. The method of claim 1 wherein the number of nucleotides of the intron of the gene of the test cell are not determined and not compared, and wherein the reference cell-line is at least partly determined by the presence and/or absence of a the gene in the test cell.

12. The method of claim 11 where the gene is an attacin gene or fragment thereof.

13. The method of claim 12 wherein the attacin gene is PPATT.

14. The method of claim 5 wherein the PCR amplifications are multiplexed.

15. The method of claim 1 wherein the reference cell-lines to be identified are selected from the group comprising MRC-5, Vero, Hi-5, CHO, FRHL2, CEF and MDCK.

16. A kit for cell-line tying comprising oligonucleotide, primers, and/or probes complementary to the calmodulin gene, the Axl receptor tyrosine kinase gene and optionally the attacin gene.

17. A kit comprising primers comprising any one of SEQ ID NO: 1, SEQ ID NO: 2, SEQ ID NO: 3, SEQ ID NO: 4, SEQ ID NO: 5 or SEQ ID NO: 6 or functional derivative thereof.

18. (canceled)
